(19) United States
(12) Patent Application Publication
(10) Pub. No.: US 2006/0172997 A1
(43) Pub. Date: Aug. 3, 2006

(54) Combinations of inhibitors of reverse transcriptase and inhibitors of virus-encoded DNA polymerase

(75) Inventors: Klaus Hamprecht, Tuebingen (DE); Herbert Schott, Tuebingen (DE); Elfriede Mikeler, Tuebingen (DE); Gerhard Jahn, Rottenburg (DE)

Correspondence Address: MORRISON & FOERSTER LLP, 12531 HIGH BLUFF DRIVE, SUITE 100, SAN DIEGO, CA, 92130-2040, US

(73) Assignee: Eberhard-Karls-Universitaet Tuebingen Universitaetsklinikum, Tuebingen (DE)

(21) Appl. No.: 11/265,825

(22) Filed: Nov. 3, 2005

Related U.S. Application Data
(63) Continuation of application No. PCT/EP04/04693, filed on May 4, 2004.

(30) Foreign Application Priority Data
May 5, 2003 (DE) 103 21 905.6

(51) Int. Cl. A61K 31/551; A61K 31/522; A61K 31/513

(52) U.S. Cl. 514/220; 514/263.32; 514/269

(57) ABSTRACT

A method for treating viral diseases which are caused by DNA viruses is described comprising administering an effective amount of at least one inhibitor of reverse transcriptase (RTI) in combination with at least one inhibitor of viral DNA polymerase, wherein the at least one RTI and the at least one DNA polymerase inhibitor are present in the form of separate compounds.

### CROSS-REFERENCES TO RELATED APPLICATIONS

This application is a continuation of copending international patent application PCT/EP2004/004693 filed on May 4, 2004, and designating the U.S., which was not published under PCT Art. 21(2) in English, and claims priority of German patent application DE 103 21 905.6 filed on May 5, 2003, which is incorporated herein by reference.

### BACKGROUND OF THE INVENTION

1. Field of the Invention

The present invention relates to antiviral substances for treating viral diseases which are caused by DNA viruses.

The search for antiviral active compounds is rendered more difficult, in particular, by the obligatory cell parasitism of the viruses. Since viruses use cellular enzymes and metabolic pathways, their genetic information is restricted to a minimum. For antiviral chemotherapy, this means that inhibition of viruses is usually associated with substantial cell toxicity.

Accordingly, only a few of the steps in viral replication are at present being used as points of attack for antiviral chemotherapeutics. An important point of attack is the inhibition of nucleic acid synthesis. In this connection, use is made, in particular, of nucleoside analogues which viral polymerases accept as substrate in place of the physiological building blocks. Incorporation of the nucleoside analogues into the growing DNA chain gives rise to enzyme blockade and thus to termination of the DNA synthesis.

The danger of contracting viral infections is particularly high in the case of immunosuppressed patients, that is, for example, following the transplantation of solid organs, bone marrow or peripheral stem cells, as well as in patients suffering from AIDS. In addition to the human immunodeficiency virus (HIV), members of the Herpesviridae family, in particular, play an important role in infectious diseases. This group includes, for example, herpes simplex virus (HSV), varicella zoster virus (VZV), Epstein-Barr virus (EBV) and human cytomegalovirus (HCMV).

2. Related Prior Art

At present, three antiviral active substances are available for being used in diseases which are caused by HCMV: ganciclovir (GCV) and ganciclovir derivatives, foscarnet (sodium phosphonoformic acid, PFA) and cidofovir (CDV).

Ganciclovir is an acyclic guanosine analogue while cidofovir is an acyclic dCMP analogue. The substances ultimately stop viral DNA synthesis by inhibiting the DNA polymerase. In order to inhibit this enzyme, ganciclovir and cidofovir have in each case to be phosphorylated to form the triphosphate. The incorporation of GCV-TP and CDV-TP into the newly forming viral DNA strand leads to the chain termination of viral DNA replication, with only ganciclovir having to be monophosphorylated by a virus-encoded enzyme.

Foscarnet on the other hand blocks the pyrophosphate-binding site of the viral DNA polymerase directly and thereby inhibits the elimination of pyrophosphate from dNTPs.

However, a disadvantage associated with administering said compounds is that the increased employment of these DNA polymerase inhibitors both in vitro and in vivo is giving rise to the formation of a large number of active compound-resistant viral strains. Mutations which arise in the target enzymes, that is, for example, the DNA polymerase, in response to the use of said substances usually constitute the molecular biological basis for the resistances. The mutations in this connection are usually characteristic for the individual classes of antiviral compounds or even for the individual compounds.

Thus, the HCMV-encoded enzyme phosphotransferase (UL-97) is, for example, mutated in ganciclovir-resistant HCMV strains. This enzyme is involved in the first step of phosphorylating ganciclovir. Mutants which are resistant to cidofovir and foscarnet usually also exhibit mutations in the viral DNA polymerase gene (for a review, see, for example, Erice, “Resistance of human cytomegalovirus to antiviral drugs”, Clin. Microbiol. Rev. 1999, 12:286-297).

At present, foscarnet is used as alternative medication when a ganciclovir-resistant HCMV infection has been detected. A disadvantage of this procedure is, however, that a relatively high dose of foscarnet is required in order to achieve an efficient antiviral effect. However, a high dose in turn results in toxic side effects.

The situation is particularly critical when multiresistance to ganciclovir, foscarnet and cidofovir is manifested in connection with HCMV infections, for example.

Furthermore, in addition to developing resistances, the toxicity of chemotherapeutics plays an important role in their use in connection with viral diseases frequently being restricted.

Thus, the administration of ganciclovir, in particular, gives rise to a change in the number of white blood cells and frequently also a change in the number of platelets. It is also possible for anemia to occur when ganciclovir is administered over a relatively long period. Because of the hematological toxicity, the administration of a full dose of ganciclovir in combination with other myelotoxic pharmaceuticals can be life-threatening (see, for example, Crumpacker, “Ganciclovir”, N. Engl. J. Med. 1996, 335:721-731).

Cidofovir exhibits a dose-dependent nephrotoxicity. This is caused by an imbalance between rapid uptake into the proximal tubule cells and the slower efflux into the urine, with this leading to accumulation in the kidneys.

Foscarnet exhibits substantial renal and metabolic toxicity. The nephrotoxicity is based on direct damage to the renal tubules.

### SUMMARY OF THE INVENTION

The present invention is therefore based on an object of making available antiviral substances which preferably provide high activity at a low dose and, furthermore preferably, in the case of resistant and multiresistant viral strains as well.

According to the invention, this object is achieved by providing a method for treating diseases and/or infections which are caused by DNA viruses, comprising administering an effective amount of at least one inhibitor of reverse transcriptase (RTI) in combination with at least one inhibitor of viral DNA polymerase and the at least one RTI and the at least one DNA polymerase inhibitor being present in the form of separate compounds.

The object underlying the invention is in this way achieved in its entirety.

In this connection, the compounds can be sold as a mixture or as what is termed a combination preparation in which the compounds are present in separate pack units in an outer pack. However, it is also possible to sell the two compound classes in separate packs and to in each case point out, in the patient information leaflet, that the compound can be employed in combination with the other respective compound for treating DNA virus infections. In this connection, the RTI and the DNA polymerase inhibitor can even be present in a chemical compound or in a mixture with other compounds.

In their own experiments, the inventors were able to demonstrate that the combined use of different RTIs together with a DNA polymerase inhibitor exhibited a synergistic virus-inhibiting effect towards a variety of HCMV strains.

These results are surprising and were not to be expected from the knowledge available in the prior art, as will be explained below.

Reverse transcriptase is an enzyme which retroviruses require for replicating their RNA. Accordingly, as expected, reverse transcriptase inhibitors (RTI) do not exhibit any effect on DNA viruses. The inventors were also able to confirm this finding, which is known in the prior art, in their own experiments.

However, entirely unexpectedly, a direct comparison of the effect of DNA polymerase inhibitors on their own with the effect of combinations of DNA polymerase inhibitors and reverse transcriptase inhibitors enabled a synergistic effect to be observed in the case of the latter. According to this comparison, the effect of the combinations on DNA viruses was markedly superior to that achieved by the individual use of the inhibitors. This manifested itself not least in the fact that it was in each case possible to employ smaller quantities for achieving an optimal effect when using a combination of the two substance classes than when using a single substance.

Consequently, contrary to the scientific findings, it was possible to demonstrate a synergistic effect of substances whose combined use in connection with diseases which are caused by DNA viruses constitutes a completely novel therapeutic approach. This is because, on the one hand, DNA viruses do not require the target enzyme of the RTIs, which means that an amplification of the effect of the DNA polymerase inhibitors, which were already being employed in the prior art, was not expected, either. On the other hand, it was additionally possible to demonstrate that it is possible, as a result of this synergistic effect, to use smaller quantities of the substances to be employed than usual in the chemotherapy.

However, as a result, completely novel antiviral active compound combinations consisting of RTIs which are per se known and DNA polymerase inhibitors which are per se known, are available which means that the side effects which are in each case possible for the individual patient can be minimized.

In one embodiment, preference is given to administer nucleoside-analogous inhibitors of reverse transcriptase and, in particular, inhibitors which are selected from the group comprising 3′-azido-2′,3′-dideoxythymidine (AZT), 2′,3′-dideoxyinosine (ddI), 2′,3′-didehydro-2′,3′-dideoxythymidine (d4T), (−)-β-L-3′-thia-2′,3′-dideoxycytidine (3TC) and 2′,3′-dideoxycytidine (ddC).

In their own experiments, the inventors were able to demonstrate that, in combination with a DNA polymerase inhibitor, these reverse transcriptase inhibitors exhibited a virus-inhibiting effect towards different HCMV strains which was increased as compared with that achieved when using the DNA polymerase inhibitors on their own.

These nucleoside-analogous or nucleosidal RTIs are commonly termed NRTIs. This substance class appears very similar to the deoxynucleotides, that is the “genuine” building blocks of the DNA. However, if the reverse transcriptase incorporates NRTIs into the developing complementary virus DNA (cDNA), it is blocked and the replication of the virus is stopped.

AZT (trade name: Zidovudine), ddI (trade name: Didanosine), ddC (trade name: Zalcitabine), d4T (trade name: Stavudine) and 3TC (trade name: Lamivudine) are NRTIs which have to be converted by cellular enzymes into the corresponding 5′-triphosphate derivatives before they are able to inhibit the virus-encoded reverse transcriptase. The substances interact with the substrate-binding site of the reverse transcriptase and inhibit virus replication by competitively inhibiting the incorporation of the natural dNTP substrates, on the one hand, and, on the other hand, precisely by their being incorporated into the growing viral DNA chain.

The incorporation of NRTIs into the viral DNA necessarily leads to chain termination since the NRTIs do not possess any 3′-hydroxyl group, which would be necessary for further extension of the DNA chain, in the sugar moiety.

Said NRTIs are adequately described in the prior art and are employed in HIV therapy. In the present instance, they can be employed in free form and/or in chemically derivatized form.

While it is true that, in addition, Kong et al., “Synergistic inhibition of human immunodeficiency virus type 1 replication in vitro by two-drug and three-drug combinations of 3′-azido-3′-deoxythymidine, phosphonoformate, and 2′,3′-dideoxythymidine”, Antimicrob. Agents Chemother. 1991, 35(10):2003-2011, have demonstrated a synergistic effect of AZT, PFA and ddT towards the retrovirus HIV, this is obvious since the substances AZT and ddT are inhibitors of the retrovirus-specific enzyme reverse transcriptase. The use of said combinations in connection with DNA viruses is not obvious precisely because of the reverse transcriptase specificity of the inhibitors.

In another embodiment, preference is given to administer nucleotide-analogous inhibitors of reverse transcriptase and, in particular, TDF (tenofovir disoproxil fumarate) and adefovir.

Nucleotide-analogous or nucleotidal RTIs are termed NtRTIs and, like the nucleoside-analogous RTIs, resemble the deoxynucleotides and are incorporated into the growing DNA strand in place of the physiological building blocks, with this leading to chain termination.

Preference is furthermore given, in another embodiment, to administer non-nucleoside-analogous and/or non-nucleotide-analogous inhibitors of reverse transcriptase.

In addition to nucleoside-analogous and nucleotide-analogous RTIs, non-nucleoside-analogous RTIs (NNRTIs), which adhere directly to the reverse transcriptase, and in this way inhibit its activity, are also known in the prior art.

Some NNRTIs, such as nevirapine, delaviridine and efavirenz, are known in the prior art. These substances bind to the reverse transcriptase and thereby inactivate it. Said substances are at present being used in the treatment of HIV infections, in particular in combination with other RTIs.

At present, combinations of nucleoside-analogous or nucleotide-analogous reverse transcriptase inhibitors, non-nucleoside-analogous reverse transcriptase inhibitors and protease inhibitors (PIs) are commonly employed within the context of successful HIV therapies (HAART: highly active antiretroviral therapy) (see, for example, Squires, “An introduction to nucleoside and nucleotide analogues”, Antivir. Ther. 2001; 6(3):1-14; Joly et al., “NNRTI plus PI combinations in the perspective of nucleoside-sparing or nucleoside-failing antiretroviral regimens”, AIDS Rev. 2002; 4:18-39; Menendez-Arias, “Targeting HIV: Antiretroviral therapy and development of drug resistance”, Trends Pharmacol. Sci. 2002; 23:381-388; De Clerq, “The Role of non-nucleoside reverse transcriptase inhibitors (NNRTIs) in the therapy of HIV-1 infections”, Antiviral Res. 1998; 38:153-179).

Although combinations of, in particular, different RTIs are known in the field for treating HIV infections, administering different RTIs and DNA polymerase inhibitors for treating diseases which are caused by DNA viruses has thus far neither been described nor rendered obvious in the prior art.

In a preferred embodiment, inhibitors of the virus-encoded DNA polymerase which are selected from the group comprising ganciclovir (GCV), foscarnet (PFA) and cidofovir (CDV) are administered.

Said DNA polymerase inhibitors are adequately described in the prior art. GCV and CDV are used, in particular, in connection with treating diseases caused by HCMV whereas PFA is also used in connection with treating viral diseases which are caused by HIV, HSV or VZV.

Since an increased use of GCV, PFA and CDV frequently leads to the development of resistant viral strains and said substances are toxic, when being used at higher doses in particular, administering, according to the invention, of combinations of these substances with RTIs offers the advantage that it is also possible to administer lower doses of these DNA polymerase inhibitors. This is made possible by the synergistic effect of the two substance classes, even when using lower doses. The risk of toxic side effects can accordingly be lowered markedly.

It will be understood, within the context of the method according to the invention, that, in addition to the possibility of combining only one RTI and one virus-encoded DNA polymerase inhibitor, it is also possible to administer two or more compounds of the two substance classes as a combinatorial pharmaceutical for treating viral diseases which are caused by DNA viruses. In the combinatorial administration of 3 or more compounds, 2 or more inhibitors in a combination can be chemically bonded to each other. The chemical bond can be effected as demonstrated, for example, in patent application WO 00/34298. In this connection, the dose quantity which is normally employed in the combination for a representative of the RTIs can, for example, be markedly reduced, with the quantity of the dose of the DNA polymerase inhibitor remaining the same.

According to a preferred embodiment of the invention, the combinations are administered for treating viral diseases which are caused by hepatitis C and/or hepatitis B viruses.

It is known from the prior art that, for example, PFA possesses a virus-inhibiting effect in connection with HBV and HCV infections. Because of the comparable mechanisms of action, the inventors assume that the combinations are also effective in the case of these viruses. Accordingly, the virus-inhibiting effect can be further amplified by combining, for example, PFA with an RTI.

In addition, the combinations can be administered for treating viral diseases which are caused by adenoviruses.

Adenoviruses cause a variety of clinical pictures, such as infections of the airways, of the eye and of the intestinal tract, which can have severe consequences in children, in particular. At present, adenoviral infections are treated, in particular, with DNA polymerase inhibitors, which means that in this case, too, because of similar mechanisms of action, the virus-inhibiting effect can be augmented by the combination with an RTI.

The combinations can furthermore be administered for treating infectious diseases which are caused by human beta-herpes viruses and/or gamma-herpes viruses and, in particular, by Epstein-Barr virus and/or human cytomegalovirus (HCMV).

People who are at particular risk of contracting severe HCMV diseases are HIV-infected patients in whom the function of the immune system has been destroyed. Patients who require medicinal immunosuppression after a transplantation of solid organs are likewise endangered, particularly in the first three months. Furthermore, recipients of bone marrow or stem cell transplantations run a high risk of suffering from an HCMV disease up until the time when the immune system which was transferred by the transplantation has assumed its full functional capacity.

In particular new infections with HCMV, or the reactivation of an infection, can lead to very severe generalized infections, with a lethal outcome, in the case of patients whose immune system is suppressed. The administering of the combinations in the case, for example, of AIDS patients suffering from an HCMV infection is therefore advantageous since two pathogens can thereby be attacked simultaneously.

It has been known for a relatively long time that ganciclovir and foscarnet, that is two DNA polymerase inhibitors, exhibit synergism in vitro in relation to HCMV replication (see, for example, Manischewitz et al., “Synergistic Effect of Ganciclovir and Foscarnet on Cytomega-lovires Replication in vitro”, Antimicrob. Agents Chemother. 1990; 34: 373-375). However, nothing has previously been known about the in-vitro synergism of NRTIs and PFAs with regard to wild-type HCMV strains and GCV-resistant HCMV UL97 mutants.

Preference is furthermore given to the combination being administered for treating viral diseases which are caused by human ganciclovir-sensitive and/or human ganciclovir-resistant cytomegalovirus strains.

Preference is furthermore given to the combination being administered for treating viral diseases which are caused by human multiresistant cytomegalovirus strains.

The combinations according to the invention provide novel possibilities for treating HCMV infections. Accordingly, it is possible, for the first time, to treat GCV-resistant and multiresistant HCMV infections antivirally in an effective manner.

In this connection, it will be understood that, when treating infectious diseases which are caused by resistant/multiresistant HCM viruses, the DNA polymerase inhibitor administered in the combinatorial pharmaceutical is PFA.

While it is true that Zaknun et al., “Concurrent Ganciclovir and Foscarnet treatment for cytomegalovirus encephalitis and retinitis in an infant with acquired immunodeficiency syndrome: case report and review”, Pediatr. Infect. Dis. J. 1997, 16:807-811 report a clinical improvement in an HCMV infection in connection with an existing HIV infection after enlisting PFA during treatment with GCV, AZT and ddI, the synergetic effect of GCV and PFA was considered to be the crucial factor in this case. An indication that it was precisely the combination of RTIs and PFA which achieved the virus-inhibiting effect cannot be found in this publication, and is not rendered obvious, either, since, according to scientific understanding, it has not previously been thought possible for RTIs in combination with DNA polymerase inhibitors to have an effect on DNA viruses.

The inventors furthermore demonstrated that even small quantities of RTI and DNA polymerase inhibitor were sufficient to achieve long-lasting virostatic effects.

Toxic side effects, that is, in particular, the nephrotoxicity of the DNA polymerase inhibitors, can be reduced by reducing the doses of the active compound combinations as compared with those of the individual substances.

Another advantage of the combination treatment of a GCV-resistant HCMV infection is that the invention can be implemented clinically immediately since the individual components have already been introduced into the respective antiviral treatment of HIV and/or HCMV.

In this connection, the pharmaceutical combinations can be administered either parenterally and/or orally.

Furthermore, the combinations can be administered together with one or more pharmaceutically acceptable excipients.

In this connection, “excipients” or auxiliary substances are understood as being those substances which are customarily used in pharmaceutical production as diluents, binders, suspending agents, lubricants, stabilizers, etc., either as a solution or as a solid substance. By way of example, these substances include, but are not restricted thereto, water, salt solutions, alcohols, vegetable oils, polyethylene glucols, gelatin, lactose, glucose, amylose, cellulose, glycerol, magnesium stearate, albumin, monoglycerides, diglycerides, polydinylpyrolidone, etc. A number of other suitable substances can be found in A. Kibbe, Handbook of Pharmaceutical Excipients, 3. ed., 2000, American Pharmaceutical Association and Pharmaceutical Press.

The pharmaceutical preparation can be sterilized, if desired, and can also comprise preservatives, stabilizers and/or dispersants, emulsifiers, buffers, dyes, flavorings, etc. which do not interfere with the active compound.

Pharmaceuticals for oral administration can be present in the form of, for example, tablets, capsules or lozenges, or as liquid preparations in the form of a syrup, which are produced using known methods, for example while adding carbohydrate binders, such as corn starch, etc. For a parenteral administration, the substance can be formulated as an injection or infusion, likewise using known methods.

In this connection, the active compound can also, for example, be incorporated into liposomes and/or nanoparticles, with the lipophilic residues which have in each case been introduced having a decisive influence on the size and stability of the liposomes.

In addition to this, the active compound can also be administered in connection with a therapeutic application in combination with other active compounds or pharmaceuticals such as reverse transcriptase inhibitors, protease inhibitors, interferon or interleukin II, or in connection with immunomodulating therapies such as bone marrow or stem cell transplantations/transplantations of solid organs, or together with active compounds which increase the number of lymphocytes. It is also possible to administer several nucleoside-phosphonoformic acid derivatives simultaneously in a formulation.

In this connection, the precise dose depends on the route of administration, the condition to be treated, the severity of the disease, the weight and age of the patient to be treated, etc.

It will be understood that the features which are described above and which are still to be explained below can be used not only in the combination which is in each case specified but also in other combinations or else on their own without departing from the scope of the present invention.

Further advantages ensue from the example.

### EXAMPLE

A cell-adapted plaque reduction assay was used for the antiviral testing (see Prix et al., “A Simplified Assay for Screening of Drug Resistance of Cell-Associated Cytomegalovirus Strains”, J. Clin. Virol. 1998; 11: 29-37). By way of example, the following virus strains were employed in this connection:

“Isolate 3”:
GCV-resistant HCMV strain, Cys603Trp mutation in the UL 97 gene, from the Gesellschaft für Virologie [Virology Society] interlaboratory experiment “GCV resistance 2000”;

GCV ID₅₀ (μM): 20.1

PFA ID₅₀ (μM): 154

“MM159838”:
HCMV strain from a transmitter's maternal milk taken on day 31 after the birth. Wild-type strain, UL97 wild-type sequence (Cys603);

GCV ID₅₀ (μM):
<6 (by definition)

PFA ID₅₀ (μM):
74.2-201.4; <400 (by definition)

By way of example, combinations of NRTIs in conjunction with the DNA polymerase inhibitor PFA were employed using the following final virostatic concentrations:

PFA: Measurement range from 100 μM to 1000 μM

AZT/3TC/ddC/d4T/ddI: from 2.5 μM to 50 μM

Cell-Adapted Plaque Reduction Assay

A modified cell-adapted plaque reduction assay was carried out for demonstrating the antiviral activity of different combinations of RTIs and DNA polymerase inhibitors. Cocultures of infected and uninfected human foreskin fibroblasts (HFFs) were used for this purpose.

The implementation of the assay was described for the first time in Prix et al., A simplified assay for screening of drug resistance of cell-associated cytomegalovirus strains, Journal of Clinical Virology 1998, 11: 29-37 and Prix et al., Comprehensive restriction analysis of the UL97 region allows early detection of ganciclovir-resistant human cytomegalovirus in an immunocompromised child, Journal of infectious diseases 1999, 180: 491-495, and is briefly described below.

Preparation of the HFF Cells

Primary HCMV isolates on HFF cells in tube culture were transferred to small cell culture flasks (25 cm²) containing a thick HFF cell lawn, and cultured. After an approx. 20-40% cytopathic effect (CPE) had been reached, the cells were washed with phosphate-buffered sodium chloride solution (PBS) and the cell lawn was detached by trypsinization. After the trypsin had been inactivated, the pellet was taken up in Eagle's minimal essential medium (MEM)-10% FCS (fetal calf serum) and filtered under mild conditions in order to separate off cell aggregates.

In parallel, the uninfected HFF cells were cultured on 175 cm² dishes, washed, detached by trypsinization and, after further purification steps, taken up in MEM-10% FCS.

Preparation of the Cocultures

In order to prepare cocultures, the infected cells were mixed with the uninfected cells in the following manner: 2.5×10⁴ uninfected cells/100 μl were mixed, in four different ratios, with 500, 1000, 2500 and 5000 infected cells/100 μl.

In each case 100 μl of each of these dilution steps were loaded, in triplicate determinations, onto a 96-well microtiter plate. The cocultures were incubated for at least 6 h, to allow the cells to adhere to the bottom of the microtiter plate, before the medium was carefully removed.

Loading on the Virostatic Agents

After incubating at 37° C. for 1 hour, 100 μl of the appropriate virostatic agent dilutions (sample mixtures) or culture medium were added. In each case 24 microcultures were carried through without virostatic agents (virus controls for the 100% plaque formation reference value). In addition, at least three microculture replicas of PFA and the respective NRTI (that is in each case as individual substances which were present separately) were carried through as were three replicas of the coculture of PFA+NRTI (toxicity control).

The cultures were subsequently incubated for up to six days such that the viruses were able to spread from the individual infected cells, in the form of plaque formation, by infecting the neighboring cells.

Detecting HCMV by in-situ ELISA

Following on from the incubation period, the medium was poured off carefully and the microcultures were permeabilized and fixed with 200 μl of ice-cold (−20° C.) methanol. After 20 min, the methanol was removed and the cells were washed twice with PBS.

The primary antibody (anti-IE1-pp72, E13 clone, Paesel & Lorei, Germany), which is directed against the HCMV immediate early antigen (UL 123) of HCMV, was then loaded on. After 1 h of incubation, the cells were washed and incubated for 1 h with the secondary antibody (horseradish peroxidase-conjugated mouse HCMV IgG antibody; DAKO, Denmark). After the peroxidase/H₂O₂ substrate had been added, the HCMV immediate early antigen was detected under the microscope in the nuclei of infected fibroblasts by means of the in-situ brown staining. The result was documented photographically in selected examples.

In the microscopic image, infected fibroblast nuclei appeared brown. It is only the plaque formation which reflects the complete virus replication in all three phases (immediate early, early and late). Stained nuclei which are solitary after five days of in-vitro culture reflect abortive infections (virus expression remained restricted to the IE stage).

In order to ensure that the evaluation was correct, a plaque had to consist of a group of at least 10 individual cells in order to be included in the count. In addition, the only dilution steps to be included were those in which there were between 15 and 60 plaques in the control series without any virostatic agent. The statistical evaluation was effected using the SPSS (statistical software package, SPSS, Munich) software to carry out a nonlinear regression analysis by means of Probit analysis. This determined the concentration of the compound which was required in order to reduce the number of plaques by 50% (inhibitory dose 50%, i.e. ID₅₀ values).

### RESULTS

The primary data obtained from the plaque reduction assay using AZT and PFA are given for the virus strain MM159838 (GCV-sensitive HCMV strain) in table 1 below.

TABLE 1
GCV-sensitive HCMV strain from maternal milk

| | AZT | | | | | | PFA | | | | | | AZT + PFA | | | | | |
|---|---|---|---|---|---|---|---|---|---|---|---|---|---|---|---|---|---|---|
| | 1 | | 2 | | 3 | | 4 | | 5 | | 6 | | 7 | | 8 | | 9 | |
| A | K | | K | | K | | K | | K | | K | | K | | K | | K | |
| | 83 | | 83 | | 81 | | 85 | | 75 | | 85 | | 104 | | 90 | | 72 | |
| B | 50 | μM | 50 | μM | 50 | μM | 1000 | μM | 1000 | μM | 1000 | μM | 50 | μM + | 50 | μM + | 50 | μM + |
| | | | | | | | | | | | | | 1000 | μM | 1000 | μM | 1000 | μM |
| MW | 49 | | 46 | | 44 | | 14 | | 16 | | 14 | | 2 | | 1 | | 0 | |
| | | | 46.3 | | | | | | 14.7 | | | | | | 1 | | | |
| C | 25 | μM | 25 | μM | 25 | μM | 500 | μM | 500 | μM | 500 | μM | 25 | μM + | 25 | μM + | 25 | μM + |
| | | | | | | | | | | | | | 500 | μM | 500 | μM | 500 | μM |
| MW | 68 | | 57 | | 59 | | 16 | | 19 | | 14 | | 4 | | 4 | | 3 | |
| | | | 61.3 | | | | | | 16.3 | | | | | | 3.7 | | | |
| D | 12.5 | μM | 12.5 | μM | 12.5 | μM | 400 | μM | 400 | μM | 400 | μM | 12.5 | μM + | 12.5 | μM + | 12.5 | μM + |
| | | | | | | | | | | | | | 400 | μM | 400 | μM | 400 | μM |
| MW | 60 | | 61 | | 61 | | 20 | | 21 | | 14 | | 5 | | 6 | | 8 | |
| | | | 60.7 | | | | | | 18.3 | | | | | | 6.3 | | | |
| E | 10 | μM | 10 | μM | 10 | μM | 300 | μM | 300 | μM | 300 | μM | 10 | μM + | 10 | μM + | 10 | μM + |
| | | | | | | | | | | | | | 300 | μM | 300 | μM | 300 | μM |
| MW | 59 | | 67 | | 68 | | 24 | | 20 | | 17 | | 11 | | 8 | | 8 | |
| | | | 64.7 | | | | | | 20.3 | | | | | | 9 | | | |
| F | 5 | μM | 5 | μM | 5 | μM | 200 | μM | 200 | μM | 200 | μM | 5 | μM + | 5 | μm + | 5 | μM + |
| | | | | | | | | | | | | | 200 | μM | 200 | μM | 200 | μM |
| MW | 63 | | 72 | | 60 | | 25 | | 26 | | 23 | | 8 | | 13 | | 9 | |
| | | | 65.0 | | | | | | 24.7 | | | | | | 10.0 | | | |
| G | 2.5 | μM | 2.5 | μM | 2.5 | μM | 100 | μM | 100 | μM | 100 | μM | 2.5 | μM + | 2.5 | μM + | 2.5 | μM + |
| | | | | | | | | | | | | | 100 | μM | 100 | μM | 100 | μM |
| MW | 85 | | 78 | | 85 | | 43 | | 43 | | 45 | | 29 | | 27 | | 33 | |
| | | | 82.7 | | | | | | 43.7 | | | | | | 29.9 | | | |

| H | K | K | K | K | K | K | K | K | K |
|---|---|---|---|---|---|---|---|---|---|
| | naw | 78 | 76 | 86 | 84 | 91 | 98 | 79 | 95 |

| | |
|---|---|
| 85.7 | (n = 23) |

In the table, the left-hand three columns show measurements made with different concentrations (50 μM to 2.5 μM) of the substance AZT on its own, while columns 4 to 6 show measurements made with different final concentrations of the substance PFA (1000 μM to 100 μM) on its own, and the measurements made for different final concentrations of the two substances in combination are given in columns 7 to 9. The table shows the number of plaques for the individual experimental assays (in each case 3 assays/test), with the mean values (MVs) for the 3 experimental assays in each case being given in the second column below the value determined for that column (that is in column 2 (B2-G2) for AZT on its own, in column 5 (B5-G5) for PFA on its own and in column 8 (B8-G8) for the combination). The final concentration (μM) of the substances used in the test is in each case given above the number of plaques which was determined. In addition, the controls (no virostatic agents, mean value H1) are in each case given in lines A and H. The virus strain used was MM 159838 (see above).

As can be seen from the table, the combination of AZT and PFA gives a markedly better reduction in the plaques than do the individual substances on their own. Thus, for example, when 12.5 μM AZT was used in the presence of 400 μM PFA (see, in each case, D7, D8 and D9), the number of plaques was reduced to an average of 6.3 as compared with the average plaque number of 60.7 when the same quantity of AZT was used on its own and with an average plaque number of 18.3 when the same quantity of PFA was used on its own. The ID₅₀ for AZT on its own was 67.0 μM (95% confidence interval, CI: 27-24574), while it was 74.2 μM for PFA on its own (CI: 29.8-116.1). In the combination, the ID₅₀ for AZT was 1.0 μM (CI: 0.4-1.7) while that for PFA was 56.2 μM (CI: 29.9-80.8).

This impressively demonstrates the synergistic effect of the substances, which means that it is also accordingly possible to markedly reduce the quantities of virostatic agents to be employed as compared with the quantities of the individual substances employed. In vivo, the quantities to be employed can in each case be matched to the patient with regard to the patient's height and weight and the severity of the disease. In every case, however, smaller quantities can be employed when the substances are combined than would be possible when using the substances individually.

In further experiments, the in-vitro synergism of the combination of AZT and PFA was also tested in regard to a GCV-resistant HCMV strain (“isolate 3”, see above). The ID₅₀ values (μM) and the 95% confidence interval (CI) were determined in three independent experiments. The results of these tests are listed in table 2 below:

TABLE 2
GCV-resistant HCMV strain
ID₅₀ (μM)

| Single component (μM) | | | | Active compound combination (μM) | | | |
|---|---|---|---|---|---|---|---|
| 2.5-50 100-1000 | | | | 100-1000 2.5-50 | | | |
| AZT | CI | PFA | CI | AZT + PFA | CI | AZT + PFA | CI |
| 59.3 | 32.5-241.0 | 126.7 | 80.1-173.5 | 88.1 | 45.8-124.2 | 1.8 | 0.7-3.0 |
| 166.0 | 64.5-2567.1 | 160.0 | 127-195.0 | 73.6 | 35-108.4 | 1.4 | 0.5-2.4 |
| 94.9 | 62.2-221.7 | 114.2 | 90-135.5 | 106.4 | 80.3-129 | 2.5 | 1.7-3.2 |

As can be seen from the table, the ID₅₀ value for AZT was markedly lower in the combination (AZT+PFA) than when AZT was used as an individual substance. In addition, it was found that the combination of NRTI and DNA polymerase inhibitor can be used to achieve a virus-inhibiting effect even on ganciclovir-resistant HCMV strains.

The effect increase index, which was calculated as follows: SI_{AZT}=ID₅₀AZT/ID₅₀ (AZT+PFA), was 32.9, 118.9 and 38.0 for AZT, based on the three independent experiments. This index describes the factor by which the virostatic effect of the single substance was increased in the combination.

In a second experimental approach, the substance d4T was combined with PFA. The experiments were performed as explained above. The primary data from the plaque reduction assay are given in table 3 below:

TABLE 3
GCV-sensitive HCMV strain from maternal milk

| | d4T | | | | | | PFA | | | | | | d4T + PFA | | | | | |
|---|---|---|---|---|---|---|---|---|---|---|---|---|---|---|---|---|---|---|
| | 1 | | 2 | | 3 | | 4 | | 5 | | 6 | | 7 | | 8 | | 9 | |
| A | C | | C | | C | | C | | C | | C | | C | | C | | C | |
| | 80 | | 79 | | 82 | | 74 | | 78 | | 82 | | 78 | | 74 | | 84 | |
| B | 50 | μM | 50 | μM | 50 | μM | 1000 | μM | 1000 | μM | 1000 | μM | 50 | μM + | 50 | μM + | 50 | μM + |
| | | | | | | | | | | | | | 1000 | μM | 1000 | μM | 1000 | μM |
| MW | 77 | | 76 | | 74 | | 11 | | 9 | | 11 | | 1 | | 0 | | 0 | |
| | | | 75.7 | | | | | | 10.3 | | | | | | 0.3 | | | |
| C | 25 | μM | 25 | μM | 25 | μM | 500 | μM | 500 | μM | 500 | μM | 25 | μM + | 25 | μM + | 25 | μM + |
| | | | | | | | | | | | | | 500 | μM | 500 | μM | 500 | μM |
| MW | 72 | | 68 | | 81 | | 12 | | 15 | | 21 | | 7 | | 4 | | 3 | |
| | | | 73.7 | | | | | | 16.0 | | | | | | 4.7 | | | |
| D | 12.5 | μM | 12.5 | μM | 12.5 | μM | 400 | μM | 400 | μM | 400 | μM | 12.5 | μM + | 12.5 | μM + | 12.5 | μM + |
| | | | | | | | | | | | | | 400 | μM | 400 | μM | 400 | μM |
| MW | 69 | | 75 | | 82 | | 10 | | 16 | | 13 | | 6 | | 6 | | 7 | |
| | | | 75.3 | | | | | | 13.0 | | | | | | 6.3 | | | |
| E | 10 | μM | 10 | μM | 10 | μM | 300 | μM | 300 | μM | 300 | μM | 10 | μM + | 10 | μM + | 10 | μM + |
| | | | | | | | | | | | | | 300 | μM | 300 | μM | 300 | μM |
| MW | 88 | | 88 | | 72 | | 16 | | 20 | | 20 | | 13 | | 9 | | 14 | |
| | | | 82.7 | | | | | | 18.7 | | | | | | 12.0 | | | |
| F | 5 | μM | 5 | μM | 5 | μM | 200 | μM | 200 | μM | 200 | μM | 5 | μM + | 5 | μM + | 5 | μM + |
| | | | | | | | | | | | | | 200 | μM | 200 | μM | 200 | μM |
| MW | 80 | | 76 | | 84 | | 25 | | 27 | | 34 | | 30 | | 30 | | 22 | |
| | | | 80 | | | | | | 28.7 | | | | | | 27.3 | | | |
| G | 2.5 | μM | 2.5 | μM | 2.5 | μM | 100 | μM | 100 | μM | 100 | μM | 2.5 | μM+ | 2.5 | μM + | 2.5 | μM + |
| | | | | | | | | | | | | | 100 | μM | 100 | μM | 100 | μM |
| MW | 79 | | 88 | | 66 | | 48 | | 38 | | 40 | | 38 | | 41 | | 38 | |
| | | | 77.7 | | | | | | 42.0 | | | | | | 39.0 | | | |

| H | C | C | C | C | C | C | C | C | C |
|---|---|---|---|---|---|---|---|---|---|
| MW | Naw | 102 | 79 | 85 | 87 | 79 | 87 | 100 | 75 |

| | |
|---|---|
| 82.3 | n = 21 |

In this case, too, as in the tests with AZT, the virus strain employed was the strain MM15938, which is a GCV-sensitive HCMV strain derived from maternal milk. In the table, the three left-hand columns show the measurements made using different final concentrations (50 μM to 2.5 μM) of the substance d4T on its own, while columns 4 to 6 show the measurements which were made with different final concentrations (1000 μM to 100 μM) of the substance PFA on its own and columns 7 to 9 give the measurements that were made for the combinations of different final concentrations of the two substances. It can be seen from these data as well that combining the two substances brought about a plaque reduction which was markedly improved as compared with that obtained when using the single substances. Thus, for example, a reduction in the plaques down to an average of 6.3 can be observed when using 12.5 μM d4T together with 400 μM PFA whereas, at the same concentration, the substance d4T only reduced the plaques down to an average of 75.3 and, at a concentration of 400 μM, the substance PFA only reduced the plaques down to an average of 13.0.

In this experimental approach, the ID₅₀ for d4T on its own was >>50 μM while that for PFA was 92.5 μM (CI: 51.1-129.8). In the combination, the ID₅₀ for d4T was 2.5 μM (CI: 1.7-3.2) and that for PFA was 105.4 μM (CI: 79.9-128.0).

In further experiments, the in-vitro synergism was also tested for the substance combinations d4T/PFA, 3TC/PFA, ddC/PFA and ddI/PFA in regard to a GCV-resistant HCMV strain (“isolate 3”, see above) and a GCV-sensitive HCMV strain. The ID₅₀ values (μM) which were determined are given in table 4 below.

In the table, “>>50” denotes that it was not possible to measure any detectable plaque reduction while “Tox” denotes that, while there was no measurable plaque reduction at 2.5 μM, the substance was toxic from a concentration of 5 μM and upwards.

TABLE 4
ID₅₀ (μM)

| Virus- | Single component (μM) | | | Active compound combination (μM) | | | |
|---|---|---|---|---|---|---|---|
| strain | 2.5-50 100-1000 | | | 100-1000 2.5-50 | | | |
| | d4T | PFA | CI | d4T + PFA | CI | d4T + PFA | CI |
| Isolate 3 | >>50 | 114.2 | 90.0-135.5 | 99.9 | 34.1-151.3 | 2.2 | 0.3-4.2 |
| MM159838 | >>50 | 92.5 | 51.1-129.8 | 105.4 | 79.9-128 | 2.5 | 1.7-3.2 |
| | 3TC | PFA | CI | 3TC + PFA | CI | 3TC + PFA | CI |
| Isolate 3 | >>50 | 114.2 | 90.0-135.5 | 108.0 | 91.8-151.8 | 2.5 | 1.6-3.4 |
| MM159838 | >>50 | 153.9 | 78.4-216.1 | 144.0 | 77.8-200 | 3.6 | 1.2-6.0 |
| | ddC | PFA | CI | ddC + PFA | CI | ddC + PFA | CI |
| Isolate 3 | 36.8 | 114.2 | 90.0-135.5 | 84.1 | 55.0-109.7 | 1.8 | 1.0-2.6 |
| MM159838 | >>50 | 153.9 | 78.4-216.1 | 144.0 | 77.8-200 | 3.6 | 1.2-6.0 |
| | ddI | PFA | CI | ddI + PFA | CI | ddI + PFA | CI |
| Isolate 3 | Tox | 114.2 | 90.0-135.5 | 54.3 | 23.8-83.7 | 0.9 | 0.004-2.5 |
| MM159838 | >>50 | 201.4 | 71.9-310 | 147.1 | 58.7-216.2 | 3.6 | 0.4-7 |

All the NRTI substances exhibited an inhibitory effect in combination with PFA both with regard to the GCV-sensitive HCMV strain and with regard to the GCV-resistant HCMV strain. It can be furthermore seen from the table that the ID₅₀ value which was determined for the NRTI in the combination was in each case markedly lower than when the single substance was used on its own.

The inventors were accordingly able to provide detailed evidence that it was possible to achieve a virus-inhibiting effect with regard to DNA viruses when combinations of selected RTIs and DNA polymerase inhibitors were used.

Therefore, what is claimed, is:

1. A method for treating diseases and/or infections which are caused by DNA viruses, comprising administering an effective amount of at least one inhibitor of reverse transcriptase (RTI), being administered in combination with at least one inhibitor of viral DNA polymerase and the at least one RTI, and the at least one DNA polymerase inhibitor being present in the form of separate compounds.

2. The method as claimed in claim 1, wherein nucleoside-analogous inhibitors of reverse transcriptase are administered.

3. The method as claimed in claim 2, wherein nucleoside-analogous inhibitors of reverse transcriptase which are selected from the group consisting of 3′-azido-2′,3′-dideoxythymidine (AZT), 2′,3′-dideoxyinosine (ddI), 2′,3′-didehydro-2′,3′-dideoxythymidine (d4T), (−)-β-L-3′-thia-2′,3′-dideoxycytidine (3TC) and 2′,3′-dideoxycytidine (ddC) are administered.

4. The method as claimed in claim 1, wherein nucleotide-analogous inhibitors of reverse transcriptase are administered.

5. The method as claimed in claim 4, wherein nucleotide-analogous inhibitors of reverse transcriptase which are selected from the group consisting of TDF (tenofovir disoproxil fumarate) and adefovir are administered.

6. The method as claimed in claim 1, wherein non-nucleoside-analogous inhibitors of reverse transcriptase are administered.

7. The method as claimed in claim 6, wherein non-nucleoside-analogous inhibitors of reverse transcriptase which are selected from the group consisting of nevirapine, delavirdine and efavirenz are administered.

8. The method as claimed in claim 1, wherein inhibitors of the virus-encoded DNA polymerase which are selected from the group comprising ganciclovir, foscarnet (PFA) and cidofovir are employed.

9. The method as claimed in claim 2, wherein inhibitors of the virus-encoded DNA polymerase which are selected from the group comprising ganciclovir, foscarnet (PFA) and cidofovir are employed.

10. The method as claimed in claim 3, wherein inhibitors of the virus-encoded DNA polymerase which are selected from the group comprising ganciclovir, foscarnet (PFA) and cidofovir are employed.

11. The method as claimed in claim 4, wherein inhibitors of the virus-encoded DNA polymerase which are selected from the group comprising ganciclovir, foscarnet (PFA) and cidofovir are employed.

12. The method as claimed in claim 5, wherein inhibitors of the virus-encoded DNA polymerase which are selected from the group comprising ganciclovir, foscarnet (PFA) and cidofovir are employed.

13. The method as claimed in claim 6, wherein inhibitors of the virus-encoded DNA polymerase which are selected from the group comprising ganciclovir, foscarnet (PFA) and cidofovir are employed.

14. The method as claimed in claim 7, wherein inhibitors of the virus-encoded DNA polymerase which are selected from the group comprising ganciclovir, foscarnet (PFA) and cidofovir are employed.

15. Method for treating diseases and/or infections which are caused by DNA viruses, comprising the step of administering an effective amount of at least one inhibitor of reverse transcriptase (RTI) in combination with at least one inhibitor of viral DNA polymerase and the at least one RTI and the at least one DNA polymerase inhibitor being present in the form of separate compounds, wherein the reverse transcriptase inhibitor employed is a compound which is selected from the group consisting of 2′,3′-didehydro-2′,3′-dideoxythymidine (d4T), (−)-β-L-3′-thia-2′,3′-dideoxycytidine (3TC) and 2′,3′-dideoxycytidine (ddC) and the viral DNA polymerase inhibitor administered is a compound which is selected from the group comprising ganciclovir, foscarnet (PFA) and cidofovir.

16. Method for treating diseases and/or infections which are caused by DNA viruses, comprising the step of administering an effective amount of at least one inhibitor of reverse transcriptase (RTI) in combination with at least one inhibitor of viral DNA polymerase and the at least one RTI and the at least one DNA polymerase inhibitor being present in the form of separate compounds, wherein the reverse transcriptase inhibitor 2′,3′-dideoxyinosine (ddI) and the viral DNA polymerase inhibitor ganciclovir or cidofovir.

17. Method for treating diseases and/or infections which are caused by ganciclovir-resistant or multiresistant human cytomegalovirus strains, comprising the step of administering an effective amount of at least one inhibitor of reverse transcriptase (RTI) in combination with at least one inhibitor of viral DNA polymerase and the at least one RTI and the at least one DNA polymerase inhibitor being present in the form of separate compounds, wherein the reverse transcriptase inhibitor employed is 3′-azido-2′,3′-dideoxythymidine (AZT) or 2′,3′-dideoxy-inosine (ddI) and the viral DNA polymerase inhibitor employed is a compound which is selected from the group consisting of ganciclovir, foscarnet (PFA) and cidofovir.

18. The method as claimed in claim 9, wherein the combinations administered for treating viral diseases which are caused by hepatitis C viruses.

19. The method as claimed in claim 9, wherein the combinations are administered for treating viral diseases which are caused by hepatitis B viruses.

20. The method as claimed in claim 9, wherein the combinations are administered for treating viral diseases which are caused by adenoviruses.

21. The method as claimed in claim 9, wherein the combinations are administered for treating viral diseases which are caused by human beta-herpes virus and/or gamma-herpes virus.

22. The method as claimed in claim 9, wherein the combinations are administered for treating viral diseases which are caused by a virus which is selected from the group consisting of Epstein-Barr virus or human cytomegalovirus.

23. The method as claimed in claim 10, wherein the combinations are administered for treating viral diseases which are caused by a virus which is selected from the group consisting of Epstein-Barr virus or human cytomegalovirus.

24. The method as claimed in claim 11, wherein the combinations are administered for treating viral diseases which are caused by a virus which is selected from the group consisting of Epstein-Barr virus or human cytomegalovirus.

25. The method as claimed in claim 16, wherein the combinations are administered for treating viral diseases which are caused by human ganciclovir-sensitive cytomegalovirus strains.

26. The method as claimed in claim 16, wherein the combinations are administered for treating viral diseases which are caused by human ganciclovir-resistant cytomegalovirus strains.

27. The method as claimed in claim 16, wherein the combinations are administered for treating viral diseases which are caused by human multiresistant cytomegalovirus strains.

28. A combination preparation for treating diseases and/or infections which are caused by DNA viruses, comprising at least one inhibitor of reverse transcriptase (RTI) and at least one inhibitor of viral DNA polymerase.
